(19) United States
(12) Patent Application Publication
(10) Pub. No.: US 2007/0227539 A1
(43) Pub. Date: Oct. 4, 2007

(54) Ventilator and method for detecting an obstruction during episodes of apnea by an additional pressure increment

(76) Inventors: Matthias Schwaibold, Karlsruhe (DE); Bernhard Scholler, Karlsruhe (DE); Sergey Velykokhatko, Karlsruhe (DE)

Correspondence Address: Friedrich Kueffner, Suite 910, 317 Madison Avenue, New York, NY, 10017, US

(21) Appl. No.: 11/731,021

(22) Filed: Mar. 29, 2007

(30) Foreign Application Priority Data
Mar. 31, 2006 (DE) 10 2006 014 956.4
Feb. 10, 2007 (DE) 10 2007 006 689.0

(51) Int. Cl. A61M 16/00; A62B 7/00

(52) U.S. Cl. 128/204.21; 128/204.18

(57) ABSTRACT

A ventilator and a method for detecting an obstruction during episodes of apnea by an additional pressure increment serve to detect episodes of obstructive apnea a respiratory gas source, a connecting line, and a patient interface are used to carry out the ventilation. The respiratory gas source has a control system for generating an incremental pressure change and at least one sensor for flow measurement. The sensor is connected to an evaluation unit that evaluates the flow pattern. A comparison of the flow pattern with the pattern of a pressure increment is carried out, and a ventilation state is automatically detected on the basis of the flow pattern.

### BACKGROUND OF THE INVENTION

1. Field of the Invention

The present invention relates to a ventilator with a respiratory gas source, a connecting line, and a patient interface. The ventilator is provided with a control system for generating an incremental pressure change, and at least one sensor for flow measurement.

The invention further relates to a method for controlling a ventilator. In accordance with the method, respiratory gas from a respiratory gas source is supplied to a patient interface through a connecting line and in which an incremental pressure pattern is generated and a flow pattern is measured.

2. Description of the Related Art

Therapeutic devices available in sleep therapy, usually, with CPAP or APAP ventilation patterns, often use an oscillatory pressure pattern for the determination of presently occurring events. This oscillatory pressure pattern is generated by a pressure oscillation source integrated in addition to the respiratory gas source and is superimposed on the pressure pattern of the respiratory gas source. The signals which are sent back to the device with a sensor according to the characteristics of the airways and pulmonary parameters are supplied to an evaluation unit, and the presently occurring event (e.g., apnea, central apnea, obstructive apnea, etc.) is determined by the evaluation unit.

### SUMMARY OF THE INVENTION

It is the primary object of the present invention to develop a device that is as simple and inexpensive as possible for determining and detecting patient parameters and events.

In accordance with the invention, this object is met achieved by connecting the sensor to an evaluation unit, which evaluates the flow pattern and compares the pattern of a pressure increment with the pattern of the flow.

A further object of the present invention is to improve a method of the aforementioned type in a way that is conducive to automated apparatus control.

In accordance with the invention, this object is met by carrying out a measurement that constitutes monitoring for the presence of an obstruction after the pressure pattern has been compared with the flow pattern.

In a preferred embodiment of the invention, a single pressure source is sufficient. This single pressure source is preferably provided by a blower. In addition, a flow sensor is incorporated in the device. In an alternative embodiment, the flow can be determined from blower characteristics, such as speed or current/power consumption.

In patients with episodes of sleep apnea, periods without spontaneous respiratory activity of the patient can occur. When this occurs, the device detects it and sends a pressure surge by means of a blower or other pressure source. This pressure surge can take the form of a pressure increment with a duration of 1 second and an amplitude of 1 hPa.

In another preferred embodiment, a distinctly greater pressure increment is applied; this ensures not only improved ability to evaluate the flow response but also ventilation of the patient by a mandatory breath in the case of centrally induced breathing interruptions. The amplitude and duration of the pressure increment are automatically determined in such a way that a certain minimum respiration of the patient is ensured. This can be preset or it can be derived from the prior breathing activity of the patient. In an especially preferred embodiment, the amplitude of the pressure increment is increased if the desired expiratory volume was not attained during the preceding pressure increment.

To wash used, CO₂-enriched respiratory air out of the ventilation system, a constant opening that allows continuous leakage flow is usually located near the patient.

When obstructive apnea is present, the pressure increase mentioned above as an example is not sufficient for removing the obstruction. Accordingly, the leakage flow increases to a constant level that is elevated relative to the state that existed before the pressure increment.

At a constant leakage flow, obstructive apnea is thus present. As a response to the detection of this event, the CPAP pressure can be increased after the apnea to keep the airways open and to prevent future episodes of apnea.

When central apnea is present, respiratory activity is inhibited by the nervous system. In this case, if a pressure increment is generated, the pressure extends into the alveoli, and the lung experiences a slight inflation/expansion. This manifests itself in a changed flow pattern. On the basis of the shape of the curve, the area above the plateau can be determined as a measure of the compliance, and the height of the peak above the plateau can be determined as a measure of the resistance.

As a response to the detection of central apnea, the device can do nothing (conventional APAP), or it can adjust the CPAP pressure to a pressure level at which the patient has a minimum number of episodes of central apnea, or it can switch from CPAP mode to a ventilation mode (bilevel) to allow practical treatment of central events also to be carried out.

The additional pressure increment is preferably applied at least once per apnea episode. In the embodiment in which the pressure increment serves as a mandatory breath, the pressure increment is preferably applied repeatedly per episode of apnea at specific intervals of time.

However, this method can also be carried out in normal respiration, whether as sleep therapy, intensive ventilation, at-home ventilation, or other application for supplying respiratory gas. Further parameters can be determined by minimal pressure increments of preferably 0.5 to 2.0 mbars and a minimum duration of preferably 0.5 to 2 seconds, depending on previously recorded inspiratory and expiratory curves; this is accomplished by evaluation of the flow response.

In another preferred embodiment, the above method can also be used after the detection of flattening.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a schematic illustration of the device with a well-defined opening for generating a leakage flow.

FIG. 2 is different flow patterns in response to incremental pressure changes.

### DETAILED DESCRIPTION OF THE INVENTION

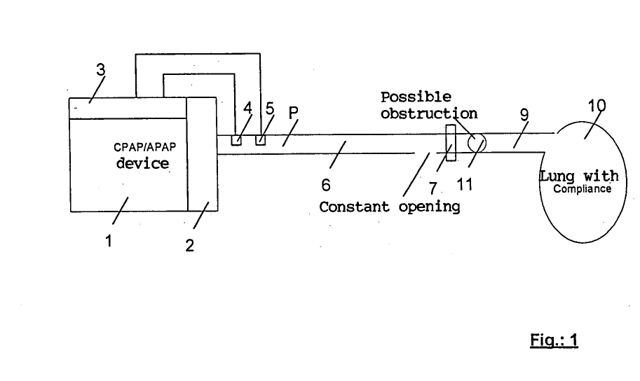

FIG. 1 is a schematic representation of a ventilator 1 with a controllable pressurized gas source 2. In the illustrated embodiment, the pressurized gas source is designed as a controllable blower with an electric drive. In addition, the ventilator 1 has an evaluation unit 3, which is connected to a pressure sensor 4 and a flow sensor 5.

The ventilator 1 is connected with a patient interface 7 by a respiratory gas line 6. A leakage opening 8 is present in the respiratory gas line 6 or the patient interface 7.

To complete the schematic representation, airways 9 and a lung 10 of a patient (not shown) are illustrated, as is an obstruction 11 in the area of the airway 9.

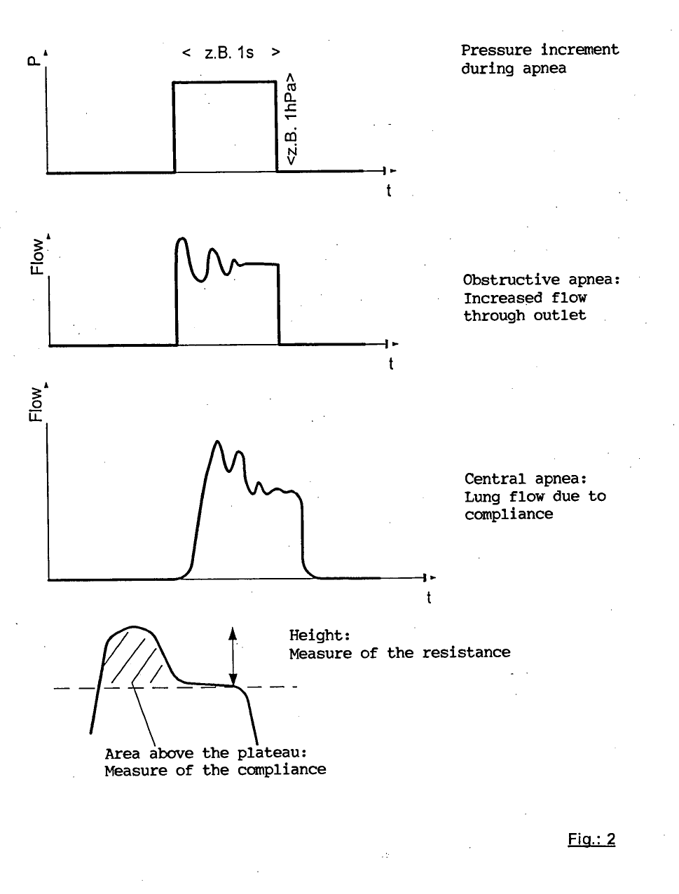

As FIG. 2 shows, there are small flow oscillations. The oscillations are caused by the system patient (airways), mask, hose, blower, pressure regulator, all of which must adjust to the new pressure. The system parameters can also be estimated from these overshoots (self-resonant frequency), e.g., from the frequency of the overshoots. This makes it possible to distinguish between central apnea and obstructive apnea.

As a specific example, the flow values shortly before the pressure jump (about 0.3 second) can be averaged to obtain an initial level, and they can be averaged at the end of the pressure jump (about the last 0.3 second) in order to determine the increased outlet flow level. The height and area of the overshoot can then be easily determined, as can the separation of the local maxima or zero crossings for overshoots in order to estimate the system parameters (see above) from the self-resonant frequency.

In a preferred embodiment, after an obstruction is detected, at least one pressure value is increased, preferably the pressure value applied at the end of the patient's expiration. In a likewise preferred embodiment, after a centrally induced cessation of breathing is detected, the difference of two pressure levels is changed; preferably, the difference between an inspiratory and an expiratory pressure level is increased.

While specific embodiments of the invention have been shown and described in detail to illustrate the inventive principles, it will be understood that the invention may be embodied otherwise without departing from such principles.

We claim:

1. A ventilator comprising a respiratory gas source, a connecting line, a patient interface, and a control system for generating an incremental pressure change, and at least one sensor for flow measurement, wherein the sensor is connected to an evaluation unit for evaluating the flow pattern and comparing the pattern of a pressure increment with the pattern of the flow.

2. A ventilator in accordance with claim 1, wherein the evaluation unit has an analyzer for carrying out an operation for detecting apnea as a function of the result of the comparison.

3. A ventilator in accordance with claim 2, wherein the analyzer is configured to distinguish obstructive apnea from central apnea as a function of the measured flow pattern.

4. A ventilator in accordance with claim 2, wherein the analyzer is designed to analyze a change in flow that occurs in response to an incremental pressure change.

5. A ventilator in accordance with claim 1, wherein the connecting line has a leakage opening.

6. A ventilator in accordance with claim 1, wherein a control system of the respiratory gas source is configured to generate a pressure surge in response to a cessation of breathing detected by the analyzer.

7. A ventilator in accordance with claim 2, wherein the analyzer is configured to detect obstructive apnea when a constant leakage flow is measured.

8. A ventilator in accordance with claim 1, wherein the control system of the respiratory gas source is configured to effect an incremental pressure increase when obstructive apnea is detected.

9. A method for controlling a ventilator, comprising supplying respiratory gas from a respiratory gas source to a patient interface through a connecting line, and generating an incremental pressure pattern and measuring a flow pattern, wherein a measurement that constitutes monitoring for the presence of an obstruction is carried out after the pressure pattern has been compared with the flow pattern.

10. A method in accordance with claim 9, wherein, on the basis of the analysis of the flow pattern, the device distinguishes between the detection of obstructive apnea and the detection of central apnea.

11. A method in accordance with claim 9, wherein a change in flow in response to an incremental pressure change is measured.

12. A method in accordance with claim 9, comprising generating a constant leakage flow.

13. A method in accordance with claim 9, comprising generating a pressure surge when cessation of breathing is detected.

14. A method in accordance with claim 9, wherein the detection of obstructive apnea is automatically generated from a constant leakage flow.

15. A method in accordance with claim 9, comprising generating an incremental pressure increase when obstructive apnea is detected.
